(19) United States
(12) Patent Application Publication
(10) Pub. No.: US 2025/0201383 A1
(43) Pub. Date: Jun. 19, 2025

(54) RECOMMENDATION BASED ON ANALYSIS OF BRAIN INFORMATION

(71) Applicant: Creator's NEXT Inc., Tokyo (JP)

(72) Inventor: Nozomu KUBOTA, Tokyo (JP)

(21) Appl. No.: 19/072,711

(22) Filed: Mar. 6, 2025

Related U.S. Application Data
(63) Continuation of application No. PCT/JP2023/034704, filed on Sep. 25, 2023.

(30) Foreign Application Priority Data
Sep. 26, 2022 (JP) 2022-152658

(51) Int. Cl. G16H 20/70; G06F 3/01; G16H 10/20; G16H 40/67

(52) U.S. Cl. CPC G16H 20/70; G06F 3/015; G16H 10/20; G16H 40/67; G06F 2203/011

(57) ABSTRACT

A method of information processing executed by one or a plurality of processors included in an information processing device which includes: generating prescribed digital data using a generator that generates digital data; acquiring a discrimination result of the prescribed digital data by inputting biometric information of a user stimulated by the prescribed digital data, the biometric information being acquired by a biometric information measuring device attached to the user, into a discriminator that uses a learning model having learned an emotion or state of the user based on the biometric information of the user through a neural network; and instructing the generator to generate digital data when the discrimination result indicates discomfort, or outputting information indicating that the prescribed digital data is comfortable for the user when the discrimination result indicates comfort.

This application is a bypass continuation of International Patent Application PCT/JP2023/034704, filed Sep. 25, 2023, which claims benefit of priority from Japanese Patent Application 2022-152658, filed Sep. 26, 2022, the contents of both of which are incorporated herein by reference.

### TECHNICAL FIELD

The present invention relates to an information processing method, a storage medium, and an information processing device capable of providing recommendations based on the analysis of brain information.

### BACKGROUND ART

Conventionally, technologies have been known that estimate users' emotions from brain wave signals and reproduce music suited to those emotions, allowing users to control their emotions and listen to pleasant music on their own (see, for example, Non-Patent Literature 1).

### CITATION LIST

### Non-Patent Literature

Non-Patent Literature 1: Ehrlich S K, Agres K R, Guan C, Cheng G (2019), “A closed-loop, music-based brain-computer interface for emotion mediation”, [online], Mar. 18, 2019, PLOS ONE, [searched on Sep. 22, 2022], on the Internet <URL: https://doi.org/10.1371/journal.pone.0213516>

### SUMMARY OF INVENTION

### Technical Problem

The conventional technologies find it difficult to estimate users' emotions from brain signals, which vary among individual users, and to appropriately estimate these emotions to provide content that suits their preferences.

Further, even though the conventional technologies estimate users' emotions from brain wave signals, they only determine whether the content being output to users is preferred and cannot generate content itself to suit individual users' preferences.

Accordingly, an object of the present invention is to provide a mechanism that can more appropriately select or generate content to suit users' preferences using data related to the brain.

### Solution to Problem

An aspect of the present invention provides a method of information processing executed by one or a plurality of processors included in an information processing device which includes: generating prescribed digital data using a generator that generates digital data; acquiring a discrimination result of the prescribed digital data by inputting biometric information of a user stimulated by the prescribed digital data, the biometric information being acquired by a biometric information measuring device attached to the user, into a discriminator that uses a learning model having learned an emotion or state of the user based on the biometric information of the user through a neural network; and instructing the generator to generate digital data when the discrimination result indicates discomfort, or outputting information indicating that the prescribed digital data is comfortable for the user when the discrimination result indicates comfort.

### Advantageous Effect of Invention

The present invention provides a mechanism that can more appropriately select or generate content to suit users' preferences using data related to the brain.

### BRIEF DESCRIPTION OF DRAWINGS

FIG. 1 is a diagram showing an example of a system configuration according to each embodiment.

FIG. 2 is a diagram showing an example of the physical configuration of the information processing device of a server according to each embodiment.

FIG. 3 is a diagram showing an example of the processing blocks of the information processing device according to a first embodiment.

FIG. 4 is a diagram showing user's states according to the first embodiment.

FIG. 5 is a diagram showing an example of association data according to the first embodiment.

FIG. 6 is a flowchart showing an example of the processing performed by the information processing device according to the first embodiment.

FIG. 7 is a diagram showing an example of the processing blocks of the information processing device according to a second embodiment.

FIG. 8 is a flowchart showing an example of the processing performed by the information processing device according to the second embodiment.

### DESCRIPTION OF EMBODIMENTS

Embodiments of the present invention will be described with reference to the accompanying drawings. Note that in the respective drawings, components denoted by the same symbols have the same or similar configurations.

### <System Configuration>

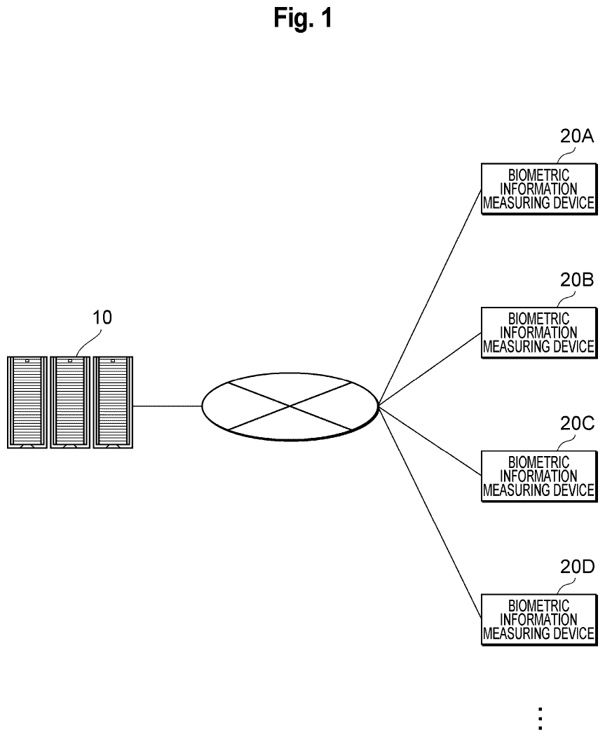

FIG. 1 is a diagram showing an example of a system configuration according to each embodiment. In the example shown in FIG. 1, a server 10 and each of biometric information measuring devices 20A, 20B, 20C, and 20D are connected via a network to allow data transmission and reception. When not separately distinguished, the biometric information measuring devices will also be referred to as biometric information measuring devices 20.

The server 10 is an information processing device capable of collecting and analyzing data and may be composed of one or a plurality of information processing devices. The biometric information measuring devices 20 are measuring devices that measure biometric information such as brain activity, heart rate, pulse, and blood flow. For example, when used as the biometric information measuring devices 20, electroencephalographs are devices equipped with invasive or non-invasive electrodes for sensing brain activity. The electroencephalographs may be any type of device, such as head-mounted or earphone types, as long as they are equipped with electrodes. The biometric information measuring devices 20 may also be devices that include the electroencephalographs and are capable of analyzing and transmitting or receiving brain information. Further, the biometric information measuring devices 20 may also be brain information measuring devices capable of performing monomolecular measurements, as will be described later.

Here, when brain activity data is used as an example of the biometric information, research has been conducted to perform machine learning on electromagnetic waveforms obtained by monomolecular measurements and to detect the single-molecule waveforms of dopamine, noradrenaline, and serotonin, which are neurotransmitters. For example, according to the paper “Time-resolved neurotransmitter detection in mouse brain tissue using an artificial intelligence-nanogap”(Yuki Komoto, Takahito Ohshiro, Takeshi Yoshida, Etsuko Tarusawa, Takeshi Yagi, Takashi Washio, & Masateru Taniguchi, “Time-resolved neurotransmitter detection in mouse brain tissue using an artificial intelligence-nanogap”, [online], Jul. 9, 2020, <https://www.nature.com/articles/s41598-020-68236-3>), three types of neurotransmitters are discriminated using a method in which the signal of an unknown sample is classified by a classifier that has learned the single-molecule waveforms of dopamine, noradrenaline, and serotonin through machine learning on electromagnetic waveforms obtained by monomolecular measurements.

According to the above brain information measuring device capable of performing monomolecular measurements, it is possible to separately measure serotonin, which generally indicates a state of calmness or the degree of relaxation, and noradrenaline, which indicates the degree of brain arousal as well as its stimulatory effects that enhance concentration or judgement. Further, it may also be possible to measure serotonin or noradrenaline using user's blood or the like other than the above brain information measuring device capable of performing monomolecular measurements.

### <Hardware Configuration>

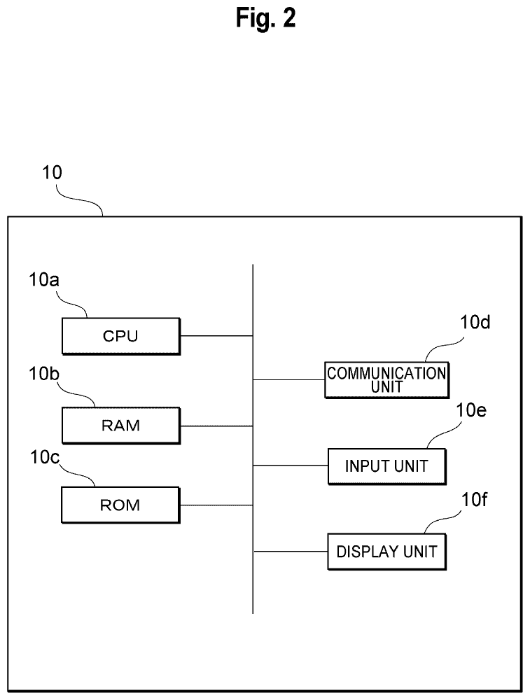

FIG. 2 is a diagram showing an example of the physical configuration of the information processing device 10 of the server according to each embodiment. The server 10 has one or a plurality of CPUs (Central Processing Units) 10a that correspond to computation units, a RAM (Random Access Memory) 10b that corresponds to a storage unit, a ROM (Read only Memory) 10c that corresponds to a storage unit, a communication unit 10d, an input unit 10e, and a display unit 10f. These configurations are connected to allow mutual data transmission and reception via a bus.

Each embodiment will describe a case in which the information processing device 10 is composed of one information processing device. However, the information processing device 10 may also be realized by combining a plurality of computers or a plurality of computation units. Furthermore, the configurations shown in FIG. 2 are provided as an example. The information processing device 10 may also have configurations other than these configurations, or it may lack some of these configurations.

The CPU 10a is a control unit that performs control related to the execution of programs stored in the RAM 10b or the ROM 10c, as well as the computation and processing of data. The CPU 10a is a computation unit that executes a program (learning program) to perform learning using a learning model that estimates user's emotions or states (for example, the degree of comport (or the degree of discomfort)) from biometric information. The CPU 10a receives various data from the input unit 10e or the communication unit 10d, and displays the computation results of the data on the display unit 10f or stores the computation results in the RAM 10b.

The RAM 10b is a storage unit in which data can be rewritten and may, for example, be composed of a semiconductor storage element. The RAM 10b may store data such as a program to be executed by the CPU 10a, data related to brain activity, and association data showing the relationships between content and indexes related to the degree of user's discomfort based on brain information. Note that such data is provided as an example. The RAM 10b may also store data other than such data or may not store some of such data.

The ROM 10c is a storage unit from which data can be read and may, for example, be composed of a semiconductor storage element. The ROM 10c may store, for example, a learning program or data that is not to be rewritten.

The communication unit 10d is an interface that connects the information processing device 10 to other equipment. The communication unit 10d may be connected to a communication network such as the Internet.

The input unit 10e receives data input from a user and may include, for example, a keyboard and a touch panel.

The display unit 10f visually displays computation results from the CPU 10a and may, for example, be composed of an LCD (Liquid Crystal Display). The display of computation results by the display unit 10f can contribute to XAI (explainable AI). The display unit 10f may also display, for example, learning results or the like.

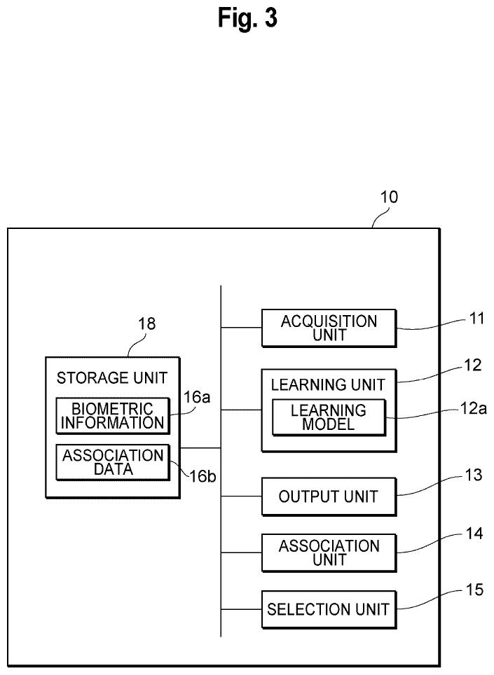

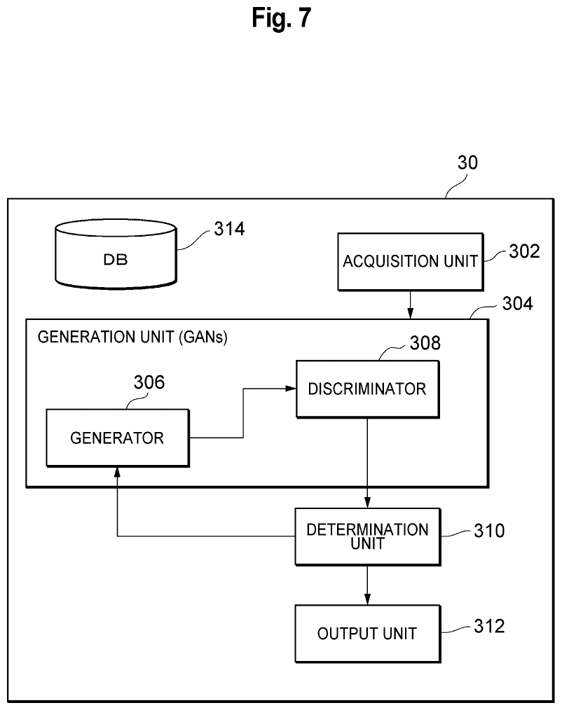

The learning program may be stored and provided on a non-transitory computer-readable storage medium such as the RAM 10b and the ROM 10c, or it may also be provided via a communication network connected via the communication unit 10d. In the information processing device 10, various operations that will be described later using FIG. 3 or FIG. 7 are realized when the CPU 10a executes the learning program. Note that these physical configurations are provided as an example and may not be necessarily independent configurations. For example, the information processing device 10 may include an LSI (Large-Scale Integration) in which the CPU 10a, the RAM 10b, and the ROM 10c are integrated. Further, the information processing device 10 may include a GPU (Graphical Processing Unit) or an ASIC (Application Specific Integrated Circuit).

### First Embodiment

Hereinafter, a first embodiment that utilizes the above-described system 1 will be described. In the first embodiment, a brain information measuring device is used as a biometric information measuring device 20, and measured data includes first data related to serotonin and second data related to noradrenaline. Additionally, serotonin and noradrenaline are neurotransmitters in the brain and can more appropriately reflect brain activity.

In the first embodiment, first data related to serotonin and second data related to noradrenaline are acquired, and user's emotions or states are estimated using training data that includes the first and second data. The user's emotions or states include, for example, whether the user feels comfort or a sense of well-being. For example, it is possible to analyze whether the user is relaxed or maintaining a calm state using the first data, and to analyze an aroused state of the brain using the second data. In the first embodiment, a calm and aroused state is defined as the user's comfort or well-being.

Furthermore, in the first embodiment, the user's brain is stimulated as content is output to the user. The content includes, for example, sounds such as music, images containing moving or still images, odors, tactile sensations, or the like. While stimulating the user's brain with the content, the first and second data are measured by the biometric information measuring device 20. By inputting the measured first and second data into a trained learning model, it becomes possible to estimate the user's emotions or states. The trained learning model includes models obtained by performing machine learning on learning models that estimate the user's emotions or states, using the first and second data as training data.

As a result, according to the first embodiment, brain activity is estimated using neurotransmitters, making it possible to more appropriately estimate the user's brain states, that is, the user's emotions or states. Further, in the first embodiment, it is also possible to provide content to the user on the basis of the estimated user's emotions or states.

### <Processing Configuration Example>

FIG. 3 is a diagram showing an example of the processing blocks of the information processing device 10 according to the first embodiment. The information processing device 10 includes an acquisition unit 11, a learning unit 12, an output unit 13, an association unit 14, a selection unit 15, and a storage unit 16. For example, the learning unit 12, the association unit 14, and the selection unit 15 shown in FIG. 3 can be executed and realized by, for example, the CPU 10a or the like, the acquisition unit 11 and the output unit 13 can be realized by, for example, the communication unit 10d or the like, and the storage unit 16 can be realized by the RAM 10b and/or the ROM 10c or the like. The information processing device 10 may be composed of a quantum computer or the like.

The acquisition unit 11 acquires first data related to serotonin and second data related to noradrenaline based on signals obtained by a biometric information measuring device 20 attached to a user while content is being output to the user. For example, the biometric information measuring device 20 acquires first data related to serotonin and second data related to noradrenaline, which are classified by a trained classifier (learning model) using an electromagnetic waveform obtained through monomolecular measurements.

The learning unit 12 performs learning of the user's emotions or states by inputting learning data including the first and second data into a learning model 12a that uses a neural network. For example, the learning unit 12 learns to output an index value that reflects a calm and aroused state using the first and second data. The learning performed by the learning unit 12 may include supervised learning using training data in which the user's emotions such as comfort, a sense of well-being, and discomfort are labeled on the basis of annotations made by the user during the measurement of the first and second data.

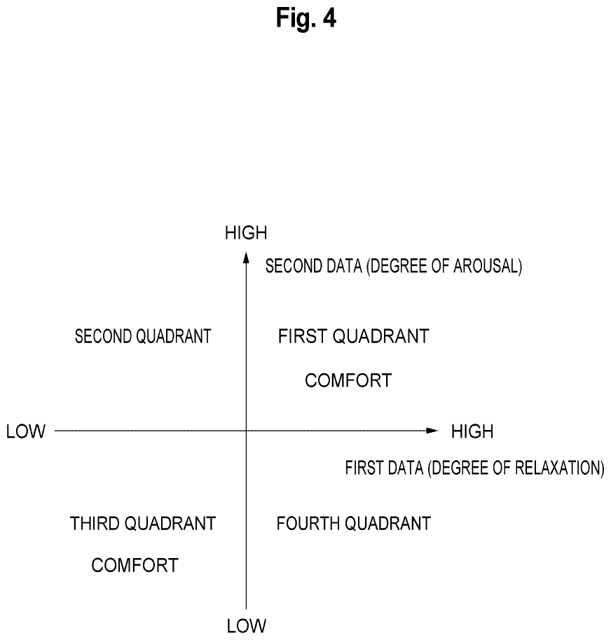

FIG. 4 is a diagram showing user's states according to the first embodiment. In the example shown in FIG. 4, the degree of relaxation is high when the first data is large, and the degree of arousal is high when the second data is large. Therefore, the first quadrant shown in FIG. 4 is defined as comfort for the user.

On the other hand, in the example shown in FIG. 4, the degree of relaxation is low when the first data is small, and the degree of arousal is low when the second data is small. Therefore, the third quadrant shown in FIG. 4 is defined as discomfort for the user. The first and second data may be determined to be large when they are equal to or more than a threshold set for each of the first and second data, or may be determined to be small when they are less than the threshold. Each threshold may be set through learning with training data in which emotions are labeled. Note that the quadrants other than the first quadrant may be defined as discomfort for the user.

Referring back to FIG. 3, the learning model 12a is a learning model that includes a neural network and includes, for example, a time series data analysis model. As a specific example, the learning model 12a may be one of a CNN (Convolutional Neural Network), an RNN (Recurrent Neural Network), a DNN (Deep Neural Network), an LSTM (Long Short-Term Memory), a bidirectional LSTM, a DQN (Deep Q-Network), or the like.

Further, the learning model 12a includes models that have been acquired through pruning, quantization, distillation, or transfer of learned models. Note that these models are provided as an example only, and the learning unit 12 may perform machine learning with other learning models.

The loss function used in the learning unit 12 includes a function that defines the degree of user's discomfort to be minimized on the basis of the first and second data. For example, as the loss function, a function is defined that minimizes the error between an index value indicating the user's comfort calculated using the first and second data and an ideal index value or annotation result corresponding to the first quadrant.

Here, the user's comfort can be defined using the first and second data. The first data is data related to serotonin, allowing for the measurement of the degree of the user's relaxation (calm state). The second data is data related to noradrenaline, allowing for the measurement of the degree of the user's arousal. For example, the loss function is set so that an index value indicating a calm and aroused state based on the first and second data increases (i.e., it is set such that the difference between the index value and an ideal index value is minimized).

Furthermore, the learning unit 12 may learn the user's emotions or states while any content is being output. For example, the learning unit 12 learns the first and second data of the user who is listening to various types of music, and then learns what type of music provides comfort to the user. Specifically, the learning unit 12 learns which type of music the user is listening to when the user's first and second data fall within the first quadrant shown in FIG. 4. As described above, when the first and second data are classified into the first quadrant, it is estimated that the user feels comfort with the music. On the other hand, when the first and second data are classified into the third quadrant (or the second or fourth quadrant), it is estimated that the user feels discomfort with the music. The learning unit 12 adjusts the bias and weights of the learning model 12a using backpropagation to minimize the output value of the loss function.

Further, the learning unit 12 may use a different learning model 12a for each user. For example, the learning unit 12 specifies a user according to user information obtained when logging into the system 1 and then performs learning using a learning model 12a corresponding to the user. As a result, by using a learning model 12a for an individual user, it becomes possible to perform learning so as to suit the user's preferences.

The output unit 13 outputs the learning result generated by the learning unit 12. For example, the output unit 13 may output the trained learning model 12a, or output an index value indicating comfort estimated by the learning model 12a or information indicating emotions or states that have been classified through learning.

The above processing allows for the provision of a mechanism that can more appropriately select or generate content to suit user's preferences using data related to the brain. For example, a learning model that can more appropriately select or generate content to suit the user's preferences can be generated using data related to the brain. Specifically, through a learning model trained with data related to serotonin and noradrenaline, it becomes possible to more appropriately estimate the user's emotions or states. Accordingly, by using this learning model, it becomes possible to provide content that more appropriately suits the user's states.

The association unit 14 associates an index value indicating the user's comfort (or discomfort) predicted by the learning of the learning unit 12 with the content that was output to the user at that time. For example, when the index value indicating the comfort included in the predicted value of a learning result exceeds a prescribed value, that is, when the user feels comfort, the association unit 14 associates information for specifying the content with the index value. As a result, by associating the index value indicating the comfort on the basis of the information of the user's brain activity with the content, a content list can be created, for example, in order of the index value indicating the comfort.

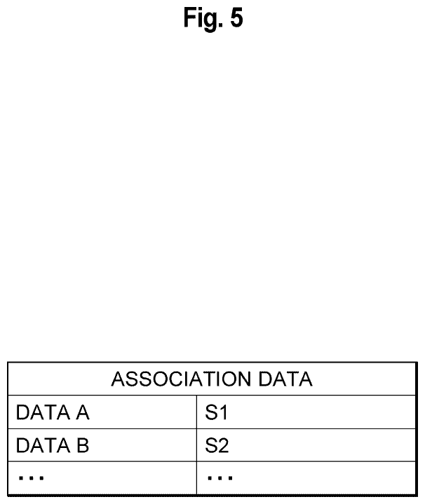

FIG. 5 is a diagram showing an example of association data according to the first embodiment. In the example shown in FIG. 5, the association data is data in which content discrimination information (for example, data A or the like) and an index value (for example, S1 or the like) are associated with each other. The association data shown in FIG. 5 is provided as an example, and may be data in which content that the user feels comfortable with and the index value at that time are associated with each other.

Furthermore, when the dataset of content that the user feels comfortable with is stored in the storage unit 16, the association unit 14 may include this content in the dataset. As a result, it becomes possible to generate a dataset in which content indicating comfort on the basis of the information of user's brain activity is aggregated.

Referring back to FIG. 3, the selection unit 15 may select at least one content from among a plurality of contents on the basis of an index value or classification result that indicates the user's comfort, which is included in the result of the learning performed by the learning unit 12. For example, when the index value or classification result indicates discomfort, the selection unit 15 selects one content from among a list of contents associated by the association unit 14 that the user feels comfortable with. Specifically, the selection unit 15 may select content in descending order of the index value (in order of comfort) or select content randomly.

In this case, the output unit 13 may output at least one content selected by the selection unit 15. The output unit 13 selects an output device according to the details of the content and outputs the content to the selected output device. For example, when the content is music, the output unit 13 selects a speaker as the output device and causes the music to be output from the speaker. Further, when the content is a still image, the output unit 13 selects the display unit 10f as the output device and causes the still image to be output from the display unit 10f.

As a result, it becomes possible to estimate the states that the user is currently experiencing on the basis of serotonin and noradrenaline, and to control the user's emotions or states to be in a more desirable way.

The storage unit 16 stores data related to the learning described above. For example, the storage unit 16 stores the information of a neural network used in a learning model, hyperparameters, and the like. Further, the storage unit 16 may also store the biometric information 16a including acquired first and second data, a trained learning model, the association data 16b shown in FIG. 5, a list of contents that the user feels comfortable with, and the like.

### Operation Example

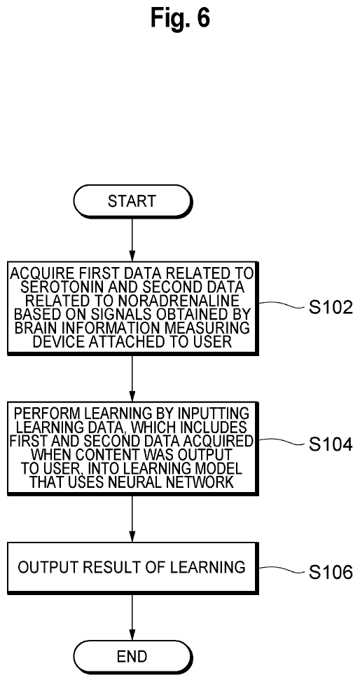

FIG. 6 is a flowchart showing an example of the processing performed by the information processing device 10 according to the first embodiment. In the example shown in FIG. 6, serotonin and noradrenaline are detected and acquired using a known technology.

In step S102, the acquisition unit 11 acquires first data related to serotonin and second data related to noradrenaline based on signals obtained by a brain information measuring device attached to a user. For example, the first data indicates the amount of serotonin secreted, and the second data indicates the amount of noradrenaline secreted.

In step S104, the learning unit 12 performs learning by inputting learning data, which includes the first and second data acquired when content was output to the user, into the learning model 12a that uses a neural network. Here, the learning model 12a is a model that learns the user's emotions or states based on the first and second data.

In step S106, the output unit 13 outputs the result of the learning obtained by the learning unit 12. The learning result may include an index value indicating the user's emotions or states. Further, the output unit 13 may output a trained model.

According to the first embodiment, by using neurotransmitters, it becomes possible to more appropriately estimate brain activity and generate a learning model that more appropriately estimates brain activity.

Further, in the first embodiment, according to the technology of the above-described “Time-resolved neurotransmitter detection in mouse brain tissue using an artificial intelligence-nanogap,” it is also possible to detect dopamine as a neurotransmitter. Therefore, the acquisition unit 11 may acquire dopamine as third data. In this case, by specifying a region in which the user feels comfort or a sense of well-being in the three-dimensional space of the first and third data, the learning unit 12 may learn the user's emotions or states on the basis of the first to third data.

Note that the acquisition unit 11 may acquire an electromagnetic waveform obtained through monomolecular measurements, and the learning unit 12 may detect dopamine, noradrenaline, and serotonin through machine learning using PUC (Positive and Unlabeled Classification) described in “Time-resolved neurotransmitter detection in mouse brain tissue using an artificial intelligence-nanogap.” Moreover, the learning unit 12 may also learn the above-described user's emotions or states by using at least the detected serotonin and noradrenaline.

### Second Embodiment

Next, a second embodiment that utilizes the same system as the above system 1 will be described. In the second embodiment, digital data being currently output to a user is regenerated using biometric information measured by a biometric information measuring device 20 so that the user feels more comfortable. The biometric information used in the second embodiment includes at least one of first data related to serotonin and second data related to noradrenaline used in the first embodiment, or data such as brain waves, blood flow, pulse, heart rate, body temperature, and eye potential.

The second embodiment uses the mechanism of a generative adversarial network referred to as a GAN. As the generator of the GAN, a generative model that generates digital data is used. As the discriminator, the learning model described in the first embodiment that estimates the user's emotions or states is used.

For example, in the second embodiment, the discriminator determines “true” when the user's emotions or states indicate comfort, and “false” when they indicate discomfort. As a result, according to the second embodiment, it becomes possible to regenerate digital data until the user feels comfortable.

### <Processing Configuration>

FIG. 7 is a diagram showing an example of the processing blocks of an information processing device 30 according to the second embodiment. The information processing device 30 includes an acquisition unit 302, a generation unit 304, a determination unit 310, an output unit 312, and a database (DB) 314. The information processing device 30 may be composed of a quantum computer or the like.

The acquisition unit 302 and the output unit 312 can be realized by, for example, the communication unit 10d shown in FIG. 2. The generation unit 304 and the determination unit 310 can be realized by, for example, the CPU 10a shown in FIG. 2. The DB 314 can be realized by, for example, the ROM 10c and/or the RAM 10b shown in FIG. 2.

The acquisition unit 302 acquires biometric information measured by the biometric information measuring device 20. The biometric information includes, for example, at least one of neurotransmitters such as dopamine, serotonin, and noradrenaline, or information such as brain waves, pulse, heart rate, body temperature, blood flow, and eye potential. Further, the acquisition unit 302 acquires the biometric information of the user stimulated using prescribed digital data. The acquisition unit 302 outputs the acquired biometric information to the discriminator 308.

The generation unit 304 generates prescribed digital data by, for example, executing the same model as a generative adversarial network (GAN). As a specific example, the generation unit 304 generates digital data that includes at least one of digital space, images including still images or moving images, music, or control signals for robots or home appliances, using a generative adversarial network (GAN) that includes the generator 306 and the discriminator 308.

The generator 306 generates digital data using input noise or the like. The noise may be a random number. For example, the generator 306 may use a neural network based on a GAN with any of the prescribed structures. Further, the generator 306 may be a generative AI that generates digital data on the basis of the input of a prompt. The generator 306 outputs the generated digital data to the discriminator 308.

The discriminator 308 acquires the biometric information of the user to whom the digital data is being output or provided from the acquisition unit 302. The discriminator 308 estimates the user's emotions or states using the acquired biometric information, with respect to the digital data generated by the generator 306. The discriminator 308 learns and discriminates the digital data as “true,” representing a positive first result, when the estimated user's emotions or states indicate comfort. On the other hand, the discriminator 308 learns and discriminates the digital data as “false,” representing a negative second result, when the estimated user's emotions or states indicate discomfort. A determination of whether the emotions or states indicate comfort or discomfort is made on the basis of the classification result when the result of the learning indicates a classification of the emotions, and on the basis of the comparison between a threshold and an index value when the result of the learning indicates the index value for the emotions or states. The discriminator 308 may also be a learning model trained with learning data that includes user's biometric information and the label of comfort or discomfort during the acquisition of the biometric information. The label of comfort or discomfort may be a label for the user's ambivalent emotions or states, such as finding something preferable or unpreferable, or finding something funny or boring.

When the result of the discrimination performed by the discriminator 308 indicates “false” (second result), the determination unit 310 instructs the generator 306 to regenerate the digital data. When the result of the discrimination indicates “true” (first result), the determination unit 310 outputs the result of the discrimination to the output unit 312. Regardless of the details of the result, the determination unit 310 may output the result of the discrimination to the output unit 312. When the generator 306 is a generative AI, the determination unit 310 may output an updated prompt to the generator 306 to generate digital data.

The generation unit 304 may update the parameters of the generator 306 and the discriminator 308 on the basis of the result of the true or false (positive or negative) discrimination performed by the discriminator 308. For example, the generation unit 304 may update the parameters of the discriminator 308 using backpropagation so that the discriminator 308 can more appropriately estimate the user's emotions or states. Further, the generation unit 304 may update the parameters of the generator 306 using backpropagation so that the discriminator 308 discriminates the digital data generated by the generator 306 as true. The generation unit 304 outputs the finally-generated digital data to the output unit 312.

When the discrimination result indicates “true” (comfort), the output unit 312 outputs information indicating that the digital data is comfort for the user. For example, the output unit 312 outputs one of a sound, an image, a mark, or the like that indicates comfort to the user so that the user can grasp his/her own state.

Further, the output unit 312 may output the digital data that the user ultimately feels comfortable with to the user. Through the above processing, it becomes possible to regenerate the digital data until the user feels comfortable.

Furthermore, the determination unit 310 may perform a determination of the discrimination result or provide an instruction to the generator 306 to generate the digital data when a prescribed condition related to the determination timing is satisfied. For example, if new digital data is generated immediately after the digital data newly generated by the generator 306 is output to the user, the user may not have enough time to experience emotions toward each piece of digital data. Accordingly, the determination unit 310 may determine the discrimination result acquired from the discriminator 308 after a prescribed time elapses, following a determination of whether the discrimination result indicates “true” or “false.”

Further, the determination unit 310 may determine whether the digital data is “true” or “false” using a plurality of discrimination results acquired within a prescribed time. For example, the determination unit 310 may employ the discrimination result that appears more frequently, or the larger of the discrimination results with the maximum absolute value of an index value indicating “true” and the maximum absolute value of an index value indicating “false” among the discrimination results acquired within a prescribed time.

Through the above processing, it is possible to provide the user with enough time to experience emotions toward each piece of digital data. Further, the user is free from feelings of urgency, unease, or doubt caused by unnecessary switching of digital data. Further, it is also possible to reduce the processing load on the information processing device 30.

Furthermore, the generated digital data may include at least one of data related to virtual space, data related to robot control, data related to autonomous driving, or data related to home appliances.

The data related to virtual space includes, for example, the metaverse and data used in the metaverse. For example, when generating the metaverse, the generator 306 is capable of continuing to generate the metaverse until the user is stimulated by the metaverse and feels comfortable when the discriminator 308 estimates the user's emotions or states.

The data related to robot control includes, for example, robots that assist human movements, nursing care robots, or the like. The user who receives services from the robot's movements feels comfort or discomfort regarding the robot's movements. For the robot's movements that the user feels uncomfortable with, the generator 306 regenerates control data to make the user feel comfortable. As a result, the generator 306 is able to continue generating control data for the robot until the user feels comfortable.

The data related to autonomous driving includes, for example, speed data of autonomous vehicles or content output inside of vehicles during autonomous driving. For example, when the generator 306 generates a moving image to be displayed inside of a vehicle during autonomous driving, the discriminator 308 estimates whether the user riding in the autonomous vehicle feels comfortable with the moving image. As a result, the generator 306 is able to continue generating moving images to be displayed inside of the autonomous vehicle until the user feels comfortable.

The data related to home appliances includes, for example, temperature control data for air conditioners. For example, when the generator 306 generates temperature control data for an air conditioner, the discriminator 308 estimates whether the user in the room where the air conditioner is placed feels comfortable with the room temperature. As a result, the generator 306 is capable of automatically adjusting the temperature of the air conditioner until the user feels comfortable.

The DB 314 stores the data processed by the generator 306 or the discriminator 308. For example, the DB 314 may store digital content generated for each user.

### <Operation>

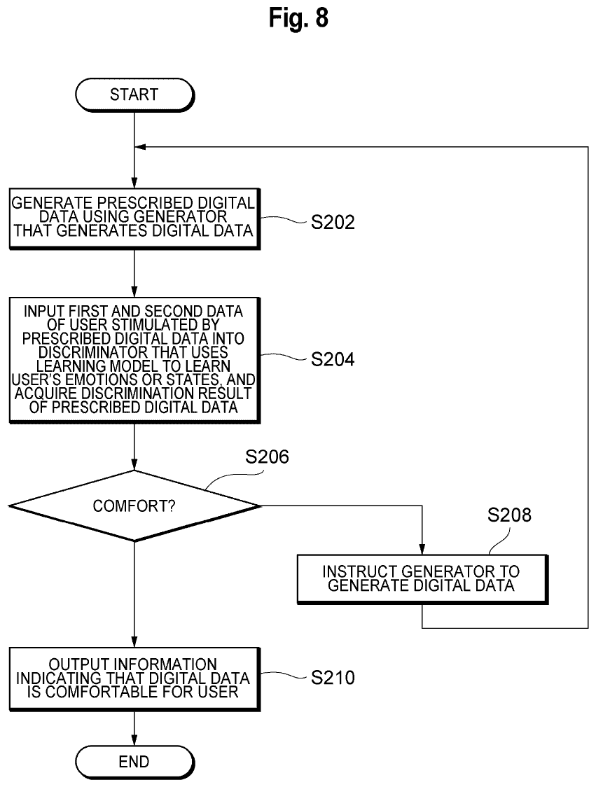

FIG. 8 is a flowchart showing an example of the processing performed by the information processing device 30 according to the second embodiment. The processing shown in FIG. 8 shows an example of continuing to generate digital data until a user feels comfortable.

In step S202, the generator 306 of the information processing device 30 generates prescribed digital data.

In step S204, the discriminator 308 of the information processing device 30 inputs the biometric information of the user stimulated by the prescribed digital data into a discriminator that uses a learning model to learn the user's emotions or states, and acquires a discrimination result that includes the user's emotions or states with respect to the prescribed digital data.

In step S206, the determination unit 310 of the information processing device 30 determines whether the discrimination result indicates comfort. For example, the processing proceeds to step S210 when the discrimination result indicates comfort (“true”) (YES in step S206), and proceeds to step S208 when the discrimination result indicates discomfort (“false”) (NO in step S206).

In step S208, the determination unit 310 of the information processing device 30 instructs the generator 306 to generate digital data. After that, the processing returns to step S202.

In step S210, the output unit 312 of the information processing device 30 outputs information indicating that the digital data is comfortable for the user.

Note that the generator 306 may be implemented in other devices instead of the information processing device 30. In this case, the information processing device 30 may output a generation instruction (for example, a prompt) to the external generator 306 and receive digital data from the generator 306.

According to the second embodiment, the above processing allows for the provision of a mechanism that can more appropriately generate content to suit user's preferences using biometric information that includes data related to the brain. Further, according to the second embodiment, it becomes possible to regenerate digital data until the user feels comfortable.

The embodiments described above aim to facilitate the understanding of the present invention and should not be used to interpret the present invention in a limiting manner. The respective elements provided in the embodiments and their arrangements, materials, conditions, shapes, sizes, or the like are not limited to those illustrated in the embodiments but can be changed as necessary. Further, it is also possible to partially replace or combine the configurations shown in the different embodiments.

### REFERENCE SIGNS LIST

10 Information processing device
10a CPU
10b RAM
10c ROM
10d Communication unit
10e Input unit
10f Display unit
11 Acquisition unit
12 Learning unit
12a Learning model
13 Output unit
14 Association unit
15 Selection unit
16 Storage unit
16a Biometric information
16b Association data
302 Acquisition unit
304 Generation unit
306 Generator
308 Discriminator
310 Determination unit
312 Output unit

1. A method of information processing executed by one or a plurality of processors included in an information processing device which comprises
generating prescribed digital data using a generator that generates digital data;
acquiring a discrimination result of the prescribed digital data by inputting biometric information of a user stimulated by the prescribed digital data, the biometric information being acquired by a biometric information measuring device attached to the user, into a discriminator that uses a learning model having learned an emotion or state of the user based on the biometric information of the user through a neural network; and
instructing the generator to generate digital data when the discrimination result indicates discomfort, or outputting information indicating that the prescribed digital data is comfortable for the user when the discrimination result indicates comfort.

2. The information processing method according to claim 1, wherein
determining whether the discrimination result is comfort or discomfort is performed when a prescribed condition related to a determination timing is satisfied.

3. The information processing method according to claim 1, wherein
the digital data includes at least one of an image, music, data related to virtual space, data related to robot control, data related to autonomous driving, or data related to a home appliance.

4. A non-transitory computer-readable storage medium having recorded thereon a program that causes one or a plurality of processors included in an information processing device to execute:
generating prescribed digital data using a generator that generates digital data;
acquiring a discrimination result of the prescribed digital data by inputting biometric information of a user stimulated by the prescribed digital data, the biometric information being acquired by a biometric information measuring device attached to the user, into a discriminator that uses a learning model having learned an emotion or state of the user based on the biometric information of the user through a neural network; and
instructing the generator to generate digital data when the discrimination result indicates discomfort, or outputting information indicating that the prescribed digital data is comfortable for the user when the discrimination result indicates comfort.

5. An information processing device including one or a plurality of processors, wherein
the one or the plurality of processors execute:
generating prescribed digital data using a generator that generates digital data;
acquiring a discrimination result of the prescribed digital data by inputting biometric information of a user stimulated by the prescribed digital data, the biometric information being acquired by a biometric information measuring device attached to the user, into a discriminator that uses a learning model having learned an emotion or state of the user based on the biometric information of the user through a neural network; and
instructing the generator to generate digital data when the discrimination result indicates discomfort, or outputting information indicating that the prescribed digital data is comfortable for the user when the discrimination result indicates comfort.
